(19) United States
(12) Patent Application Publication
(10) Pub. No.: US 2025/0244477 A1
(43) Pub. Date: Jul. 31, 2025

(54) FOUR-DIMENSIONAL ULTRASOUND IMAGING SYSTEM AND METHOD

(71) Applicant: SHENZHEN CARDIOACC LTD, Shenzhen (CN)

(72) Inventors: Xinhuan ZHOU, Shenzhen (CN); Jun MA, Shenzhen (CN); Yuqin LIU, Shenzhen (CN); Jiahui ZHAO, Shenzhen (CN); Sumin ZHUANG, Shenzhen (CN)

(73) Assignee: SHENZHEN CARDIOACC LTD, Shenzhen (CN)

(21) Appl. No.: 19/182,537

(22) Filed: Apr. 17, 2025

Related U.S. Application Data
(63) Continuation of application No. PCT/CN2023/137314, filed on Dec. 8, 2023.

(30) Foreign Application Priority Data
Oct. 11, 2023 (CN) 202311315998.X

(51) Int. Cl. G01S 15/89; B06B 1/02; G01N 29/24; G01S 7/52

(52) U.S. Cl. CPC G01S 15/8977; B06B 1/0292; G01N 29/2406; G01S 7/52028; G01S 15/8925; G01S 15/8993

(57) ABSTRACT

A four-dimensional ultrasound imaging system including an ultrasound system, a two-dimensional capacitive ultrasound transducer, and an ASIC (Application Specific Integrated Circuit) chip is introduced. Specifically, a transmission driving circuit and a receiving circuit are integrated in the ASIC for driving the transducer to transmit ultrasound beams and receive reflected echoes. The ASIC is configured to perform microchip pre-beamforming on the received signals to reduce channel number, and then the processed signals are transmitted to the ultrasound system for analog to digital conversion, beamforming and coordinate conversion. A four-dimensional ultrasound image is obtained by fast and repeatedly acquiring three-dimensional images. In the present application, the ASIC chip is integrated in the transducer, so that the two-dimensional transducer can have fewer wires. Subsequent imaging processing is performed in an ultrasound system processor to reduce wires of the transducer and the data throughput, as well as increase the data computation efficiency.

### CROSS-REFERENCE TO RELATED APPLICATIONS

This application is a Bypass Continuation of the PCT application with the international application number PCT/CN2023/137314 filed on De. 8, 2023, designating the United States, now pending, and further claims foreign priority to Chinese Patent Application No. 202311315998.X filed on Oct. 11, 2023. The entire contents of the above-mentioned applications are incorporated herein by reference.

### TECHNICAL FIELD

The present disclosure relates to the technical field of medical image, in particular to a four-dimensional ultrasound imaging system and method.

### BACKGROUND

Electrophysiological heart disease and structural heart disease are important healthcare problems. Electrophysiological heart disease is usually caused by irregular electric current transmission in the heart. Structural heart disease includes decrease in cardiac stroke volume and abnormal blood pressure due to cardiac structural abnormalities (such as myocardial hypertrophy). Currently minimally invasive catheter intervention is the safest and most effective treatment methods for most electrophysiological heart diseases and structural heart diseases. By advancing ablation catheters (e.g., microwave ablation, radiofrequency ablation, pulsed electric field ablation and cryoballoon ablation) into the cardiac chamber via peripheral vein intervention, myocardium and endocardium are ablated for eliminating diseased heart tissues and electrical conduction bundles. Real-time 4D (four-dimensional) cardiac ultrasound imaging during intervention can guide the relative positions of the catheter and the cardiac chamber in real time, thus minimizing the occurrence of surgical complications (such as thrombus and pericardial effusion), which has important clinical significance.

4D Intracardiac Echocardiography (ICE) is one of the most advanced 4D cardiac imaging technologies. By inserting a miniature ultrasound transducer into the cardiac chamber through femoral veins, real-time 4D high-resolution imaging is performed from within the heart. Existing 4D ICE uses two-dimensional phase array transducers, wherein thousands of elements are integrated in the miniaturized transducer. Each array element requires separate wiring and control. Therefore, the transducer and the ultrasound system have a large number of channels and high cost. Another challenge is that thousands of wires cannot pass through the chamber of the catheters, which has to be as small as possible to minimize the puncture damage to the patients. 4D phase array ICE usually uses a piezoelectric ceramic phase array ultrasound transducer. In order to minimize the packaging complexity and reduce the number of wires, the piezoelectric ceramic phase array ultrasound transducer is integrated into an Application Specific Integrated Circuit (ASIC) chip, and the ASIC chip preprocesses data to reduce the number of wires. However, in the current technology, data processing of the ASIC is complicated and slow.

### SUMMARY

Based on this, it is necessary to propose a four-dimensional ultrasound imaging system and method to reduce the complexity of packaging and number of wires, the channels and data amount in four-dimensional ultrasound imaging, and further improve the processing efficiency in the current technology.

On the first aspect, the present disclosure provides a four-dimensional ultrasound imaging system. The system includes an ultrasound system, a multi-channel two-dimensional phase array capacitive ultrasound transducer, and an ASIC chip integrated on a transducer side. The ASIC chip includes a transmission driving circuit and a receiving circuit. The transducer is connected with the ultrasound system through cables and a connector.

The transmission driving circuit is configured to drive each channel of the transducer so that the transducer transmits an ultrasound beam in a designated direction in the three-dimensional space.

Each channel of the transducer is configured to receive the reflected echo signal from the tissue after transmission of the beam. The receiving circuit is configured to perform preprocessing for channel reduction operation on the reflected echo signal based on a microchip pre-beamforming algorithm. After preprocessing and channel reduction, the analog signals are acquired by the ultrasound system for three-dimensional beamforming and image formation.

The ultrasound system is configured to perform beamforming operation on the reflected echo signal based on the beamforming algorithm, and then calculate the gray values of all voxels along the ultrasound beam. The ultrasound system is also configured to calculate the coordinate value of each voxel on the ultrasound beam according to echo time, beam direction and sound speed.

The ultrasound system obtains the gray values and coordinate values of all voxels along the ultrasound beams in all directions in the three-dimensional space by scanning the designated direction in the region of interest. Then the gray values and coordinate values of each voxel are converted to a Cartesian coordinate system to form a three-dimensional image.

The ultrasound system obtains dynamic three-dimensional images by acquiring multiple three-dimensional images consecutively. The ultrasound system is also configured to perform rendering and slicing of the dynamic three-dimensional image (i.e., four-dimensional ultrasound image) on a monitor.

Optionally, the two-dimensional phase array capacitive ultrasound transducer includes a large number of array elements in a two-dimensional matrix manner. Each array element includes a number of CMUT (Capacitive Micromachined Ultrasound transducers) cells which are connected in parallel. Each array element can be integrated with the ASIC chip by TSV (Through Silicon Via) technology or simple wiring.

Each CMUT cell includes an upper and a lower electrodes and silicon on insulator, and the silicon on insulator forms a vacuum by silicon wafer bonding technology. In the transmit mode, a stimulating waveform is applied between the upper and lower electrodes of each CMUT cell to form mechanical oscillation. In the receive mode, each CMUT cell undergoes mechanical oscillation upon external sound force, and the mechanical oscillation is converted into voltage signal.

The CMUT cell perforates a hole in a wafer by Through Silicon Via (TSV) technology or direct wiring to connect the upper and lower electrodes of each array element to the ASIC chip for signal connection.

Optionally, the microchip pre-beamforming algorithm includes delay and sum of signals from all channel of the transducer to obtain M analog signals. Pre-beamforming algorithm calculates the direction of the ultrasound beam, the transmit delay and the receive delay for each voxel after receiving the reflected echo signal of the transmitted ultrasound beam. M is a positive natural and is equal to the number of wires of the transducer and smaller than the number of channels of the transducer. In this way, pre-beamforming conducted with the ASIC reduces the number of wires for the transducer, and thus reducing the packaging complexity.

The beamforming algorithm is conducted with the ultrasound system. The beamforming algorithm includes delay and sum of the M analog signals, interpolation and coordinate conversion to form a three-dimensional ultrasound image.

On the second aspect, the present disclosure provides a four-dimensional ultrasound imaging method. The method is applied to the four-dimensional ultrasound imaging system in the first aspect. The method includes the following steps:

transmitting the ultrasound beam in a designated direction in the three-dimensional space in a sequence until the whole region of interest is scanned, and acquiring the reflected echo signals by the transducer;

performing preprocessing and channel reduction operation on the microchip using pre-beamforming algorithm. Then the number of received signals can be reduced by the ASIC chip to M, wherein M is the number of wires of the transducer and smaller than the number of transducer channels;

performing beamforming on the M signals by the ultrasound system to obtain the gray value and coordinate value of each voxel along the transmit beam direction;

repeatedly executing the above steps until the ultrasound system obtains the gray values and coordinate values of all voxels in the three-dimensional region of interest, and converting the gray values and coordinate values of all voxels to the Cartesian coordinate system to form a three-dimensional image;

repeatedly executing the above steps so that the ultrasound system obtains a dynamic three-dimensional image; and performing rendering and slicing operation of the four-dimensional ultrasound image on the monitor.

Optionally, before transmitting the ultrasound beam in a designated direction in the three-dimensional space, the method also includes the following steps: calculating transmit delays according to the position of the focal point and the transducer elements, and stimulating the channels of the transducer based on the transmit delays and the preset waveform, thereby generating the ultrasound beam in a designated direction;

After the ultrasound beam is transmitted into the tissue, the reflected ultrasound echoes are then received by the transducer and converted to the analog signals.

Optionally, the multi-channel two-dimensional phase array capacitive ultrasound transducer includes thousands of transducer array elements arranged in a two-dimensional grid. The calculation of the transmit delays includes the following steps:

setting the focal point for each transmit, and calculating the distances of the focal point to the active array elements in the transducer. The transmit delay for each active element is calculated by division of the distance by sound of speed;

transmitting the ultrasound beam in another direction by changing the coordinates of the imaging focal voxel and apply the updated transmit delay. Repeating the above steps until completion of the region of interest scanning.

Optionally, the two-dimensional transducer array elements are divided into M sub-groups of array elements. Pre-beamforming for channel reduction is conducted on the ASIC chip, and after pre-beamforming the number of signal channels is reduced from the number of elements to M. Pre-beamforming includes:

calculating the total delay for each transducer element. The total delay is the sum of transmit delay and receive delay. For each beam scan direction, the transmit distance for each transducer element is the distance from each transducer element to the focal point. Then the receive distance is the distance between each voxel and the transducer element. The total delay is the sum of transmit distance and receive distance divided by speed of sound;

For each transmit delay, conduct pre-beamforming to obtain M signals specifically includes: calculating the first distance from the focal point to each array element in the sub-group of array elements and a second distance from the focal point to each group's geometric center. Then the distance difference between the first distance and the second distance divided by the sound speed is the time difference for each element. After calculating the time difference for each element, applying a delay to the signal of corresponding array element, followed by summing all delayed signals within the sub-group. For the total M group, this will result in M analog signals to be transferred to the ultrasound system;

wherein M is a positive natural number and equal to the number of wires of the transducer and smaller than the number of channels of the transducer. For example, the transducer is a 64×16 two-dimensional phase array divided into 32 (M=32) sub-groups, then each group has 32 elements. The transducer is connected to the ultrasound system via connectors, and it has 32 wires. The pre-beamforming is conducted in the ASIC.

Optionally, the total delay is the summation of the transmit delay and receive delay.

Optionally, beamforming is conducted on the M signals by the ultrasound system to obtain the gray value and coordinate value of each voxel on the ultrasound beam in a certain direction specifically includes:

M analog signals are received by the ultrasound system to calculate one line of voxels along the beam direction. In this step, each sub-group of the transducer behaves as one element to the ultrasound system for three-dimensional beamforming, and its center is the same with the geometric center of the subgroup. The transducer is connected to the ultrasound system via M wires, and it behaves as a M channel transducer to the ultrasound system.

Pre-beamforming filters are applied to the M analog signals, followed by analog to digital conversion. Delay and sum and interpolation are applied to the digital signals to obtain the gray value and coordinate value of each voxel along the ultrasound beam in a certain direction.

Optionally, three-dimensional reconstruction including interpolation and the conversion of the gray values and coordinate values of all voxels to the Cartesian coordinate system are performed parallelly with multiple-threads in a GPU (Graphic Processing Unit) or CPU (Central Processing Unit).

The embodiment of the present disclosure has the following beneficial effects.

The present disclosure provides a four-dimensional ultrasound imaging system. The system includes an ultrasound system, a multi-channel two-dimensional phase array capacitive ultrasound transducer with an ASIC chip. The ASIC chip includes a transmission driving circuit and a receiving circuit. The transducer is connected with the ultrasound system through a system end connector and a catheter end connector. Specifically, a transmission driving circuit is conducted in the ASIC chip to stimulate each channel of the transducer to transmit ultrasound beams in a designated direction. After each channel of the transducer receives the reflected echo signals, pre-beamforming is conducted in the ASIC for preprocessing and channel reduction. So the number of channels in the transducer can be significantly reduced. The above steps are repeated with different beam directions until completion of scanning of the region of interest. Then a three-dimensional image will be generated, rendered and displayed on the monitor. In the present disclosure, the ASIC is integrated in the transducer to reduce the complexity and number of wire connections. Subsequent imaging processing can be performed in an ultrasound system processor, so that the packaging and wiring complexity is greatly reduced, as well as reducing the data throughput and improving post-processing efficiency.

### BRIEF DESCRIPTION OF THE FIGURES

The embodiments in the following description are merely a part rather than all of the embodiments of the present disclosure. For those skilled in the art, under the premise of without contributing creative labor, other attached figures further can be obtained according to these attached figures.

Wherein,

FIG. 1 is an internal structure diagram of a multi-channel two-dimensional phase array capacitive ultrasound transducer in an embodiment of the present disclosure.

FIG. 2 is a bonding structure diagram of one array element and the ASIC in an embodiment of the present disclosure.

FIG. 3 is a structure diagram of a four-dimensional ultrasound imaging system in an embodiment of the present disclosure.

FIG. 4 is a flow diagram of a four-dimensional ultrasound imaging method in an embodiment of the present disclosure.

FIG. 5a is a work flow diagram that an ASIC is connected with an ultrasound system processor in an embodiment of the present disclosure.

FIG. 5b is a schematic diagram of an ultrasound beam transmitted by a multi-channel two-dimensional phase array capacitive ultrasound transducer in an embodiment of the present disclosure.

FIG. 6 is a flow chart of a data preprocessing method in the ASIC chip in an embodiment of the present disclosure.

FIG. 7 is an illustrative schematic diagram of various ultrasound transducers in an embodiment of the present disclosure.

FIG. 8 is an internal structure chart of computer equipment in an embodiment of the present disclosure.

### DETAILED DESCRIPTION OF THE EMBODIMENTS

The following describes the technical solutions in the embodiments of the present disclosure with reference to the attached figure. Apparently, the described embodiments are only a part rather than all of the embodiments of the present disclosure. All other embodiments obtained by those skilled in the art based on the embodiments of the present disclosure without creative efforts shall fall within the protection scope of the present disclosure.

In the embodiment of the present disclosure, in the ICE (Intracardiac Echocardiography), a miniaturized transducer is installed in the distal tip of a cardiac catheter and then advanced to the heart chamber through peripheral veins. The transducer transmits a sound wave to scan a designated direction in a three-dimensional space, and then the reflected echo is processed by a computer to form an ultrasound image. High-resolution real-time images of intracardiac anatomical structures and other intervention catheters can be visualized in real time.

ASIC (Application Specific Integrated Circuit) is designed and manufactured for specific user requirements and specific electronic systems. The computing power and efficiency can be customized according to algorithm needs.

CMUTs (Capacitive (Micromachined Ultrasound transducers) are capacitive electrotransducers. With micromechanical technology and capacitive sensing technology, high-precision and high-sensitivity ultrasound detection and measurement can be implemented.

Flip Chip is one of chip packaging technologies. The pin points of the chip are connected downwards in the connection process that connect points of the chip to a substrate or a circuit board.

TSV is Through Silicon Via technology, and is essentially one of the above Flip Chip bonding technologies. Specifically, vertical connection is made between chips and wafers. TSV technology implements vertical electrical interconnection of silicon vias by filling conductive materials such as copper, tungsten, and polysilicon.

From the above, it is clear that the existing two-dimensional ultrasound transducer and post-processing are complicated and slow in data processing. Therefore, a novel data processing method is provided in the embodiment of the present disclosure. That is, received channel data from the transducer is preprocessed with a microchip pre-beamforming algorithm in the ASIC, and then the preprocessed data is transmitted to an ultrasound system for further imaging processing. In this way, the number of channels and the amount of data are greatly reduced, while greatly improving the data processing efficiency. At the same time, the number of wires of the transducer can be greatly reduced, so that a micro 4D ultrasound endoscope with simple packaging and wiring becomes possible. In addition, the cost of CMUT transducer is greatly reduced compared with traditional piezoelectric transducer. At the same time, the transducer is connected with the ultrasound system with fewer wires, so that the data throughput is reduced, and the data is processed more quickly.

Based on the above, the embodiment of the present disclosure first introduces a multi-channel two-dimensional phase array capacitive ultrasound transducer with ASIC, then introduces a four-dimensional ultrasound imaging system based on the ultrasound transducer, and finally proposes a four-dimensional ultrasound imaging method. Thus, the embodiment of the present disclosure reduces the cost of transducer and the complexity of wiring, and further improves the computational efficiency in four-dimensional ultrasound imaging.

### Embodiment I

Existing 3D/4D (three-dimensional/four-dimensional) ICE usually use a two-dimensional phase array or a rotating one-dimensional phase array. For the two-dimensional phase array method, it usually has thousands of elements in the phase array transducer to transmit ultrasound beams in different directions in the 3D region of interest. In endoscopic ultrasound imaging, it is necessary to reduce the number of transducer wires so that the transducer and wires can be integrated into a small catheter. It is usually necessary to integrate a disposable ASIC chip at the front end of the catheter for imaging preprocessing, resulting in high development cost of 4D piezoelectric phase array ICE and high difficulty in integration and processing.

In the embodiment of the present disclosure, a multi-channel two-dimensional phase array capacitive ultrasound transducer is proposed. The transducer includes an ASIC chip and two-dimensional elements, where each element contains a number of electrotransducer (CMUT) cells. The CMUT and the ASIC chip are bonded by TSV technology in Flip Chip or direct wiring to reduce the manufacturing difficulty. It is also expected to reduce the cost of the ultrasound transducer and catheter.

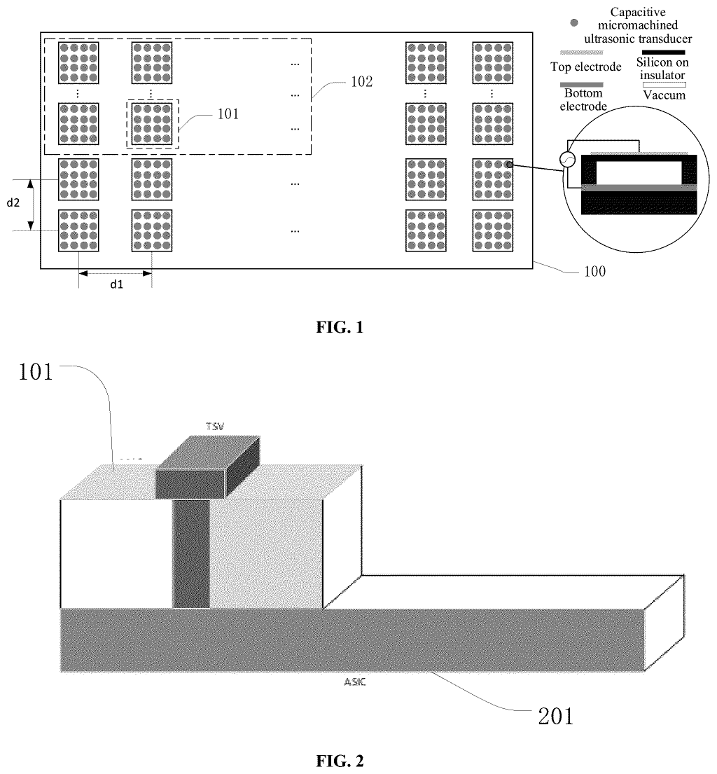

FIG. 1 is an internal structure diagram of a multi-channel two-dimensional phase array capacitive ultrasound transducer. The ultrasound transducer includes a number of array elements 101. The array elements 101 are a two-dimensional matrix. Each array element 101 includes a number of CMUT cells. The CMUT cells are connected in parallel and driven by a same excitation waveform to enhance the transmit signal sensitivity, and the signal amplitude is enhanced by parallel superposition of signals. FIG. 2 is a bonding structure diagram of one array element and the ASIC chip in an embodiment of the present disclosure. A hole is perforated in a wafer by TSV technology. The upper and lower electrodes of each array element 101 are connected to a base plate of the ASIC 201. High density integration of the ASIC chip and the CMUT is implemented. It is assumed that there are N ultrasound array elements 101 in total, there are N connection pin points connected to the ASIC chip.

Specifically, combined with FIG. 1 and FIG. 2, the multi-channel two-dimensional phase array capacitive ultrasound transducer includes an application specific integrated circuit (ASIC) chip 201 and a two-dimensional phase array transducer 100. The transducer 100 includes a number of array elements 101 divided into M sub-groups 102. Each sub-group of array elements 102 includes a number of array elements 101. Each array element 101 includes a number of capacitive electrotransducers (CMUTs) connected in parallel and driven by the same excitation waveform. The capacitive electrotransducers CMUTs are bonded and connected to the ASIC chip 201 by flip-chip packaging technology. Each capacitive electrotransducer (CMUT) includes upper and lower electrodes and silicon on insulator, and the silicon on insulator forms a vacuum in the middle by silicon wafer bonding technology. In the transmit mode, a stimulating waveform is applied between the upper and lower electrodes of each capacitive electrotransducer (CMUT) to form mechanical oscillation. In the receive mode, each capacitive electrotransducer (CMUT) undergoes mechanical oscillation under the action of an external sound field, and the mechanical oscillation is converted into voltage change. The capacitive electrotransducer (CMUT) perforates a hole in a wafer by Through Silicon Via (TSV) technology to connect the upper and lower electrodes of each array element 101 to a base plate of the ASIC chip.

In the embodiment of the present disclosure, the transducer 100 is bonded to the ASIC chip 201, wherein the ASIC chip includes both signal excitation and signal receiving. In the transmit mode, the ASIC chip applies the excitation waveform upon each array element. In the receive mode, the ASIC chip receives analog signals from all active transducer channels and performs signal preprocessing. The bonding technology between the traditional ceramic chip transducer and the ASIC is complicated and extremely high in cost. Therefore, in the embodiment of the present disclosure, the CMUT is bonded to the ASIC chip by TSV technology. Then, a number of CMUT cells are connected in parallel to enhance transmission and receiving signal amplitude. The production difficulty and cost of the four-dimensional ultrasound transducer can be significantly reduced where two-dimensional phase array CMUT cells are integrated with the ASIC chip. At the same time, compared with the existing commercial piezoelectric phase array ultrasound transducer, the ultrasound transducer in the embodiment of the present disclosure can greatly reduce the equipment cost and technological difficulty, so that low-cost large-scale production of 4D ultrasound transducers becomes possible.

### Embodiment II

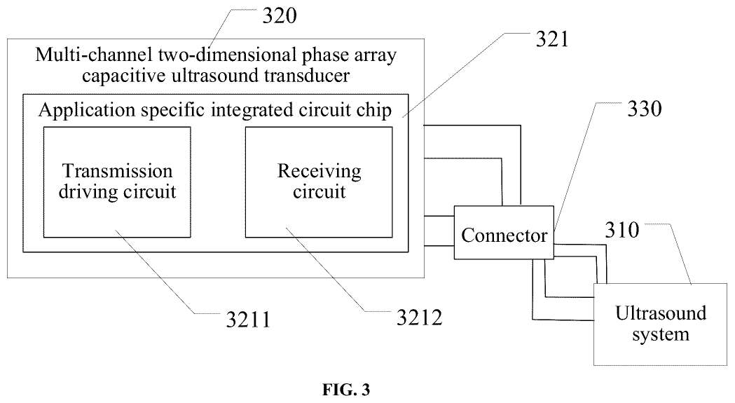

FIG. 3 is a structure diagram of a four-dimensional ultrasound imaging system according to an embodiment of the present disclosure. The system includes an ultrasound system 310, a multi-channel two-dimensional phase array capacitive ultrasound transducer 32, and an ASIC chip 321 integrated on a transducer. The ASIC chip includes a transmission driving circuit 3211 and a receiving circuit 3212. A wire of transducer 320 is connected with the ultrasound system 310 through a connector 330. In the embodiment of the present disclosure, the four-dimensional ultrasound imaging system displays four-dimensional ultrasound imaging by transmission, receiving, beamforming, image synthesis and rendering display sequentially.

Specifically, in the transmission step, the transmission driving circuit 3211 is configured to stimulate each channel of the transducer using a pre-set waveform so that the transducer 320 transmits an ultrasound beam in a designated direction, and the ultrasound beam in a designated direction is configured to scan a designated direction in a three-dimensional space.

In the receiving step, each channel of the transducer 320 is configured to receive a reflected echo signal of the ultrasound beam. The receiving circuit 3212 is configured to perform pre-beamforming, i.e., preprocessing and channel reduction operation on the reflected echo signal. The transducer 320 is also configured to transfer the reflected echo signal after pre-beamforming to the ultrasound system 310 through the connector 330.

Wherein, the transducer 320 includes a number of channels. After being transmitted, the ultrasound beam is transmitted into a tissue in the three-dimensional space and is reflected back to the transducer. It can be understood that the receiving circuit 3212 of the ASIC chip performs preprocessing with a microchip pre-beamforming algorithm, so that a large amount of channel data can be reduced and transferred to the ultrasound system 310 with fewer wires.

For each transmit event, the ultrasound system 310 performs beamforming on the signal after preprocessing and channel reduction operation, and then obtain the gray values of all voxels along the ultrasound beam in a designated direction. The ultrasound system 310 is also configured to calculate the coordinate value of each voxel on the ultrasound beam in a designated direction according to echo time, space direction and sound speed.

The transmit direction is then changed for each transmit event until the whole three-dimensional region of interest is scanned. The gray values and coordinate values of all voxels on the ultrasound beams in all directions are obtained by repeatedly changing the direction of the transmitted ultrasound beam, followed by repeating the above steps of transmission, receiving, pre-beamforming and beamforming. Then a three-dimensional image in Cartesian coordinate is formed by interpolation and coordinate conversion to obtain the three-dimensional image.

Wherein, for each transmit event, after pre-beamforming filters and analog to digital conversion the beamforming operation includes calculating the transmit delay and receive delay of each voxel, followed by interpolation and summation.

In the rendering display step, a dynamic three-dimensional image is obtained by repeatedly executing the steps of transmission, receiving, beamforming and image synthesis. Rendering and slicing operation is performed on the monitor to display a four-dimensional ultrasound image.

It should be noted that the dynamic three-dimensional image here is essentially a four-dimensional ultrasound image. Rendering, slicing and other operation are performed on the dynamic three-dimensional image in the ultrasound system, and the dynamic three-dimensional image is displayed on the monitor. It should also be noted that the rendering and slicing operation here refers to certain operation on the monitor so that the dynamic three-dimensional image can be more easily visualized on the monitor, so the technology here is not limited to only rendering and slicing, and all technologies that can achieve similar purposes can be applied to the embodiments of the present disclosure.

In the embodiment of the present disclosure, the ASIC chip is integrated in the transducer, so that the stimulating and post-processing of the multi-channel transducer can be implemented with fewer wires and thus number of signal channels. Subsequent imaging processing can be performed in an ultrasound system processor, so that the processing data is simple, the data throughput is small, and the data processing efficiency is improved.

### Embodiment III

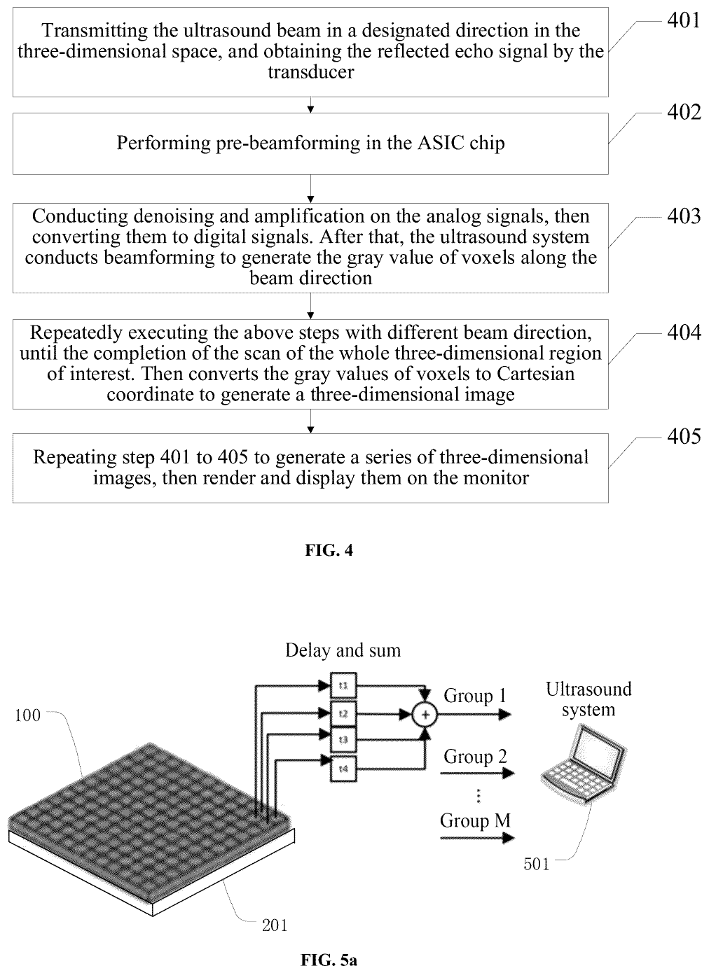

Based on the four-dimensional ultrasound imaging system, the embodiment of the present disclosure proposes a four-dimensional ultrasound imaging method. FIG. 4 is a flow diagram of a four-dimensional ultrasound imaging method in an embodiment of the present disclosure. Specifically, the method includes the following steps:

step 401, transmitting the ultrasound beam in a designated direction in the three-dimensional space, and obtaining the reflected echo signal obtained after the ultrasound beam is transmitted by the two-dimensional phase array multi-channel capacitive ultrasound transducer;

step 402, performing pre-beamforming on the reflected echo signal by the receiving circuit, i.e., preprocessing and channel reduction in the ASIC;

step 403, performing beamforming operation on the transferred analog signal after preprocessing and channel reduction operation. After beamforming, the ultrasound system obtains the gray value and coordinate value of each voxel along the transmitted ultrasound beam in a certain direction;

step 404, repeatedly executing the above steps with different transmit beam direction, until the ultrasound system completes scan of the three-dimensional region of interest;

step 405, repeatedly executing the above steps so that the ultrasound system obtains a dynamic three-dimensional image, i.e., a four-dimensional ultrasound image.

An example is, in step 402, that the ultrasound transducer includes 1024 array elements, and the ASIC chip preprocesses the received 1024 signals from all elements according to the microchip pre-beamforming algorithm, and the number M of wires is 32. Therefore, the microchip beamforming algorithm can significantly reduce the difficulty of wire bonding and packaging, reduce the size of the wire harness and the catheter, and reduce the number of system channels and the data volume.

The embodiment of the present disclosure conducts two parts of beamforming with two devices. Step 403 to step 405 is based on the ultrasound system processor, where the transducer behaves as a two-dimensional phase array transducer with M elements. The other part is the pre-beamforming in the ASIC chip, where step 401 to step 402 are conducted. This method of dividing the synthesis into two parts of “a front end (the ASIC)” and “a back end (the ultrasound system processor)” significantly reduces the amount of calculation for processing data, and improves computation efficiency.

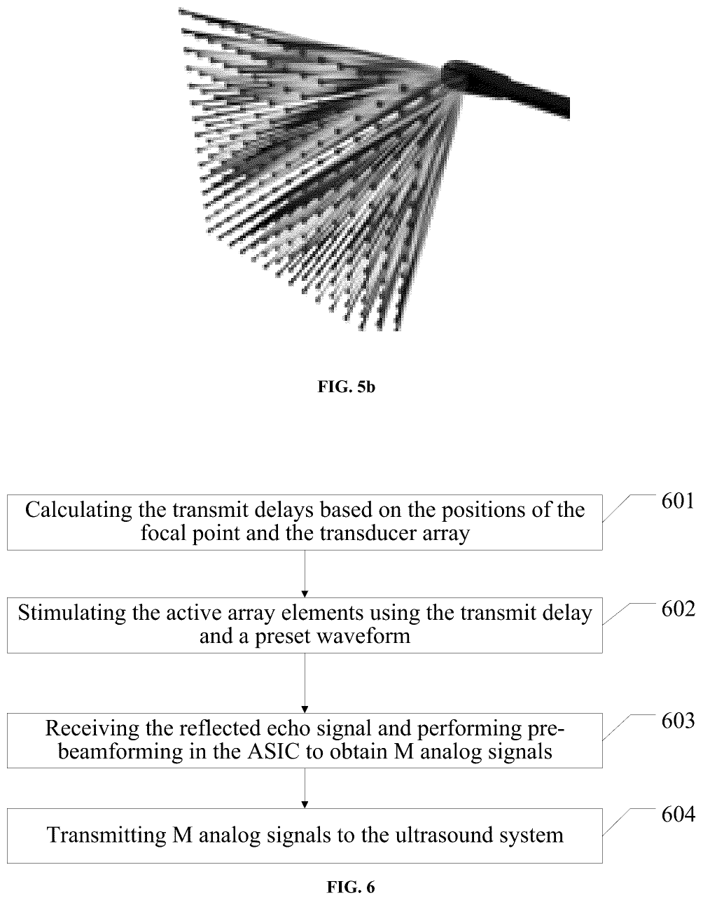

FIG. 5a is a work flow diagram and shows that an ASIC is connected with an ultrasound system processor in an embodiment of the present disclosure. The ASIC stimulates the transducer elements to scan a three-dimensional space. FIG. 5b is a schematic diagram of an ultrasound beam transmitted by the multi-channel two-dimensional phase array capacitive ultrasound transducer. After hundreds of ultrasound beams are transmitted to scan the whole region of interest, beamforming (that is, image reconstruction) is performed to obtain a three-dimensional image. In the embodiment of the present disclosure, when the multi-channel two-dimensional phase array capacitive ultrasound transducer transmits an ultrasound beam, reflected echo signal is further processed to obtain a four-dimensional image.

FIG. 6 is a flow chart of a data preprocessing method by an ASIC chip in an embodiment of the present disclosure. The method includes the following steps.

Step 601, calculating transmit delays based on the transducer and the position of the transducer array elements.

In the embodiment of the present disclosure, for each transmit beam, the focal point is determined by the beam's spatial direction and focal distance. Then the coordinates of the focal point and those of all array elements are obtained for distance calculation. Then the transmit delay for each element can be calculated by dividing the distance by speed of sound. The space direction of the ultrasound beam is repeatedly changed to complete scan of three-dimensional region of interest.

Step 602, based on the transmit delays, setting a same preset waveform to sequentially excite each active array element, thereby realizing beam scanning in any direction in the imaging space.

Step 603, receiving the reflected echo signal obtained after beam scanning, and performing pre-beamforming for channel reduction operation to obtain M signals.

For each transmit event in the thee-dimensional image scanning sequences, the direction of the transmitted beams will change to scan the full three-dimensional space. The focal point is chosen once the transmit direction and focal depth is determined, and the transmit delay for each active transducer channel is calculated as the distance of focal point to the element divided by speed of sound.

For the purpose of channel and wire number reduction, the two-dimensional phase array transducer are divided into M sub-groups of array elements. A first distance from the focal point to each array element in each sub-group is calculated, and then a second distance from the focal point to each group's geometric center is calculated. Assume the sub-group has K elements, then a number of K distance differences between the first distance and the second distance can be obtained, followed by divided by the sound speed to obtain K time delay. Then the K time delay is applied to the received signal of corresponding array element. After that, the K properly delayed analog signals are summed to obtain one analog signal. Then for M sub-groups, a total number of M analog signals will be transferred via M wires to the ultrasound system. This pre-beamforming algorithm is conducted in the ASIC chip.

Wherein, M is a positive natural number and equal to the number of transducer wires, but smaller than the number of transducer channels. In step 603, after pre-beamforming, the number of signals is reduced to M, which greatly reduces the complexity of transducer packaging and wiring, as well as improving data transfer efficiency and computational efficiency. Reducing the wires of transducer also makes it possible to miniaturize the ultrasound transducer for endoscope application, and also reduce cost and the channels of ultrasound system.

Step 604, transferring the M analog signals from the transducer to the ultrasound system through M cables and connectors. Then pre-beamforming filters are applied to the analog signals for denoising, amplification etc. Filtered analog signals are then converted to digital signals in the Analog-to-Digital-converter.

In the embodiment of the present disclosure, the beamforming in the ultrasound system is the same with existing three-dimensional ultrasound systems. The transducer behaves as a M channel transducer, where each sub-group functions as one element in the follow-up beamforming step. The ultrasound system obtains the coordinate value of each voxel in the beam direction according to the time of flight. After pre-beamforming filtering and analog-to-digital-conversion, the beamforming in the ultrasound system performs subsequent steps includes step i to step v.

i, treat the transducer as a M channel phase array transducer. According to the coordinate value of each voxel in the beam direction, calculate the transmit delay and receive delay for the M-channel transducer. The receive distance is the transmit distance from each voxel to all the M element positions, and the transmit distance is the distance from the focal point to all the M element positions. Then transmit delay and receive delay are transmit distance and receive distance divided by speed of sound.

ii, for each voxel, apply delay to M digital signals and then sum the interpolated M value from M signals for the voxel. Wherein, interpolation calculation usually requires a large amount of calculation, so multi-threaded parallel calculation may be performed in a GPU or a CPU. Then for each transmit event, the gray values of voxels along the beam direction can be calculated.

iii, repeating the above steps with different transmit directions, complete scanning of the whole three-dimensional space. Then converting the gray values in the three-dimensional scattered points to Cartesian coordinate to form a three-dimensional image.

iv, the above steps i to iii are performed repeatedly so that a series of dynamic three-dimensional images can be acquired for subsequent rendering, displaying and slicing.

It can be understood that the above operation such as interpolation, three-dimensional reconstruction, rendering, slicing, and conversion of gray values and coordinate values to the Cartesian coordinate systems can be performed in multiple threads in a GPU or a CPU to improve computational efficiency.

In the embodiment of the present disclosure, beamforming algorithm comprises two parts. One part is pre-beamforming for analog signal in the ASIC chip, and the second part is the beamforming algorithm for digital signal in the ultrasound system. Being different with existing ultrasound system where beamforming is all conducted in the ultrasound system, the present disclosure can significantly reduce the amount of data and the number of wires and channels for the ultrasound system, and can improve the post-processing efficiency.

In one embodiment, an ultrasound system processor is proposed. The ultrasound system processor stores a computer program. When the computer program is executed by the ultrasound system processor, the ultrasound system processor executes the method in any embodiment based on any one of the methods in the ultrasound system.

In one embodiment, an ASIC chip is proposed. The ASIC chip stores a computer program. When the computer program is executed by the chip, the chip executes the method in any embodiment based on the methods in the ASIC chip.

It can be understood that a complete implementation step based on the four-dimensional ultrasound imaging system and method in an actual situation is illustrated as follows: a multi-channel two-dimensional phase array capacitive ultrasound transducer is integrated with ASIC and is connected via cables and connectors to an ultrasound system. The four-dimensional ultrasound imaging algorithm includes the ASIC end pre-beamforming algorithm and the system end beamforming algorithm.

Firstly, ASIC end pre-beamforming includes the following:

I, calculation of the transmit delays. In a transmit mode, the ASIC chip calculates the transmit delays according to the distance from each array element to the focal point. Assume the index of an ultrasound array element is (i, j), its coordinates is (i*d1, j*d2, 0). Then assume the coordinate of the focal point is (Xf, Yf, Zf), the distance from each array element to the focal voxel is \sqrt{(xf−i*d1)²+(yf−j*d2)²+(zf)²}. Therefore, the transmit delay from the array element to the focal voxel is \sqrt{(xf−i*d1)²+(yf−j* d2)²+(zf)²}/c, wherein c is the sound speed, which is generally 1540 m/s in tissue.

II, transmission excitation. The ASIC chip excites each array element with the same preset waveform according to the transmit delay of each array element, and then all active array elements according to the element's corresponding transmit delay and waveform, to generate a beam scanning in the designated direction in the space.

III, ASIC end pre-beamforming algorithm. During receiving, the array elements of the ultrasound transducer are divided into M sub-groups, wherein each sub-group has a number of array elements. Assume each sub-group has N elements denoted as {T1, T2, . . . , TN}. The number of array elements in each group can be the same or different. During each transmit, the transmit distance from each array element to the focal point is calculated, and then the distance from the focal point to the geometric center of the group of array elements is calculated. The difference between the two distances divided by the sound speed is the delay, which is recorded as {τ1, τ2, . . . , τN}. The analog signals received by all active array elements in each sub-group are delayed by {τ1, τ2, . . . , τN} and then summed to obtain one signal respectively. Then, M signals are obtained and transferred to the ultrasound system through M cables.

Then, denoising filtering and analog-to-digital conversion is performed on the pre-beamformed signal.

IV, beamforming in the ultrasound system processor. The M signals are delayed and summed, and then corresponding interpolation operation is performed according to the coordinate value of each voxel in the beam transmitting direction to obtain the gray-scale map of each voxel along the beam direction. The interpolation calculation usually requires a large amount of calculation, and multi-threaded parallel calculation may be performed in a GPU or a CPU.

V, the above steps II to IV are repeated until the whole region of interest is scanned, followed by interpolation to Cartesian coordinate to form a three-dimensional image. Then, the above steps are repeatedly executed to obtain a dynamic three-dimensional image. The dynamic three-dimensional image is rendered and displayed on the monitor of the ultrasound system.

It can be understood that the above description is only an example, and does not limit scope of the technologies made based on the embodiment of the present disclosure.

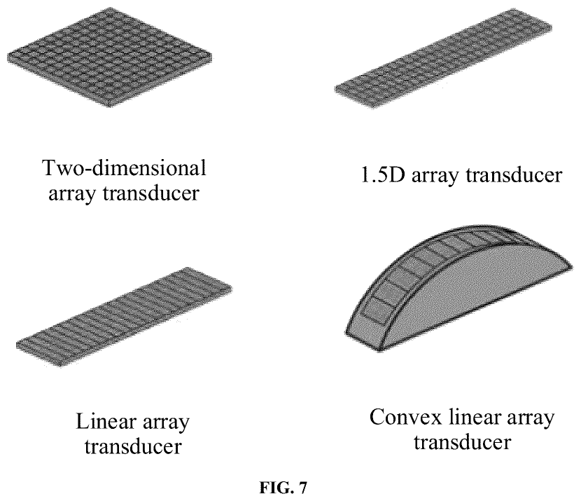

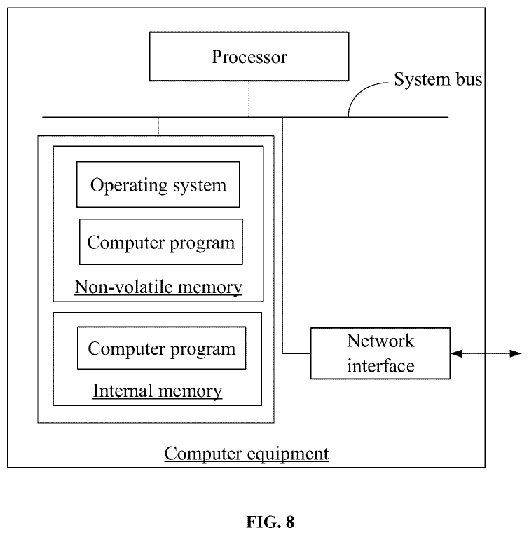

In one feasible implementation method, the transducer used in the embodiment of the present disclosure may be a two-dimensional phase array ultrasound transducer, and may be arranged in a rectangular or hemispherical shape. As shown in FIG. 7, the material of the transducer may be PZT ceramic, CMUT or PMUT. At the same time, the four-dimensional ultrasound imaging system can be applied to endoscopic ultrasound imaging such as intracardiac ultrasound, esophageal ultrasound, vaginal ultrasound, bronchial ultrasound, and can also be applied to extracorporeal transducers such as transthoracic ultrasound transducers. Further, in the solution of the embodiment of the present disclosure, an ADC analog-to-digital converter, a gain and other units may be added to the ASIC chip to improve signal quality. In addition, the present disclosure can not only be applied to a diagnostic ultrasound transducer, but also therapeutic ultrasound transducer such as focused ultrasound.

FIG. 8 is an internal structure chart of computer equipment in an embodiment of the present disclosure. The computer equipment may be a workstation, a server, or a gateway. As shown in FIG. 8, the computer equipment includes a processor, a memory, and a network interface connected by a system bus. Wherein, the memory includes a non-volatile storage medium and an internal memory. The nonvolatile storage medium of the computer equipment stores an operating system, and can also store a computer program. When the computer program is executed by the processor, the processor can realize the steps in the above method. The computer program can also be stored in the internal memory. When the computer program is executed by the processor, the processor can execute each step in the method. A person skilled in the art may understand that the structure as shown in FIG. 8 is just a block diagram of a related partial structure in the solution of the present disclosure, but does not constitute a limitation on the computer equipment applied in the solution of the present disclosure.

The program may be stored in a computer readable storage medium. When the program executes, the processes of the methods in the embodiments are performed. Wherein, any reference to a memory, storage, a database, or other media used in the embodiments provided by the present disclosure may include non-volatile and/or volatile memory. The non-volatile memory may include a read-only memory (ROM), a programmable read-only memory (PROM), an electrically programmable read-only memory (EPROM), an electrically erasable programmable read-only memory (EEPROM), or a flash memory. The volatile memory may be a random access memory (RAM) or an external cache memory. By way of illustration but not limitation, the RAM is available in various forms, such as a static RAM (SRAM), a dynamic RAM (DRAM), a synchronous DRAM (SDRAM), a double data rate SDRAM (DDRSDRAM), an enhanced SDRAM (ESDRAM), a synchronous link DRAM (SLDRAM), a memory bus (Rambus) direct RAM (RDRAM), a direct memory bus dynamic RAM (DRDRAM) and a memory bus dynamic RAM (RDRAM).

The above embodiments only express several embodiments of the present disclosure, and the description is specific and detailed, but cannot be construed as limiting the claims of the present disclosure. It should be noted that several modifications and improvements may also be made to those skilled in the art without departing from the concept of the present disclosure, which fall within the scope of the present disclosure. Therefore, the protection scope of the present disclosure shall be subject to the claims.

1. A four-dimensional ultrasound imaging system, comprising an ultrasound system, a multi-channel two-dimensional phase array capacitive ultrasound transducer, and an ASIC (Application Specific Integrated Circuit) chip integrated on a transducer side, wherein the ASIC chip comprises a transmission driving circuit and a receiving circuit; wherein the wires of the transducer are connected with the ultrasound system through the system end connector and the catheter end connector;
wherein the transmission driving circuit is configured to stimulate each channel of the transducer so that the transducer transmits an ultrasound beam in a designated direction, the ultrasound beam in a designated direction being scanned in a designated direction in a three-dimensional space;
wherein each channel of the transducer is configured to receive a reflected echo signal after the ultrasound beam is scanned, and transmit the reflected echo signal to the receiving circuit;
wherein the receiving circuit is configured to perform preprocessing and channel reduction operation on the reflected echo signal based on a microchip beamforming algorithm; wherein the transducer is also configured to transmit the reflected echo signal after preprocessing and channel reduction operation to the ultrasound system through the connector;
wherein the ultrasound system is configured to perform beamforming operation on the reflected echo signal, and then obtain the gray values of all voxels along the ultrasound beam; wherein the ultrasound system is also configured to calculate the coordinate value of each voxel along the ultrasound beam according to echo time, space direction and sound speed;
wherein the ultrasound system obtains the gray values and coordinate values of all points on the ultrasound beams in all directions in the three-dimensional space by repeatedly adjusting the designated direction and repeating the above process, the gray values and coordinate values being converted in a Cartesian coordinate system to obtain a static three-dimensional image;
wherein the ultrasound system obtains a dynamic three-dimensional image by repeating the above process; and wherein the ultrasound system performs rendering and slicing operation of the generated four-dimensional images on a monitor for clinical use.

2. The system according to claim 1, wherein the multi-channel two-dimensional phase array capacitive ultrasound transducer comprises a large number of array elements arranged in a two-dimensional matrix, each array element comprises a number of CMUTs (Capacitive Micromachined Ultrasound transducers) connected in parallel; wherein each array element is connected to the ASIC chip by TSV (Through Silicon Via) technology;
wherein each CMUT comprises upper and lower electrodes and silicon on insulator, the silicon on insulator forming a vacuum by silicon wafer bonding technology; wherein in a transmit mode, an alternating current is applied between the upper and lower electrodes of each CMUT to form mechanical oscillation; wherein in a receive mode, each CMUT cell's membrane undergoes mechanical oscillation upon external sound field, the mechanical oscillation being converted into voltage change; and
wherein the CMUT perforates a hole in a wafer by through silicon via technology to connect the upper and lower electrodes of each array element to a base plate of the ASIC chip.

3. The system according to claim 1, wherein the microchip beamforming algorithm comprises delaying and adding signals corresponding to each channel of the transducer to obtain M signals according to the direction of the ultrasound beam, a transmission time difference and a receiving time difference based on the reflected echo signal after the ultrasound beam is scanned, wherein M is a natural number of greater than zero, M being equal to the number of leads of the transducer and smaller than the number of channels of the transducer; and
wherein the pre-beamforming algorithm comprises delay and sum the M signals to obtain data after beamforming.

4. A four-dimensional ultrasound imaging method, wherein the method is applied to the four-dimensional ultrasound imaging system according to claim 1, the method comprising the following steps:
transmitting the ultrasound beam in a designated direction in the three-dimensional space, and obtaining the reflected echo signals after the ultrasound beam is transmitted by the transducer;
performing preprocessing and channel reduction operation based on the microchip beamforming algorithm on the reflected echo signal by the receiving circuit of the ASIC chip to obtain M signals, wherein M is a natural number of greater than zero, M being equal to the number of leads of the transducer and smaller than the number of channels of the transducer;
performing beamforming operation and first calculation operation based on the M signals after preprocessing and channel reduction operation by the ultrasound system to obtain the gray value and coordinate value of each point on the ultrasound beam in a designated direction;
repeatedly executing the above steps until the ultrasound system obtains the gray values and coordinate values of all voxels in all directions of the three-dimensional space, and converting the gray values and coordinate values of all voxels to the Cartesian coordinate system to obtain a three- dimensional image;
repeatedly executing the above steps so that the ultrasound system obtains a dynamic three-dimensional image composed of a series of three-dimensional images; and performing rendering and slicing operation on the monitor to obtain a four-dimensional ultrasound image.

5. The method according to claim 4, wherein before transmitting the ultrasound beam in a designated direction in the three-dimensional space by the transducer, the method also comprises the following steps: obtaining different transmit delays by the transmission driving circuit, and stimulating the channels of the transducer in sequence based on the transmit delays, thereby generating the ultrasound beam in a designated direction; and
wherein the step of obtaining the reflected echo signal obtained after the ultrasound beam is transmitted by the transducer specifically comprises: reflecting the transmitted ultrasound beam in a corresponding medium in the three-dimensional space to generate the reflected echo signal, and obtaining the reflected echo signal by the transducer.

6. The method according to claim 5, wherein the multi-channel two-dimensional phase array capacitive ultrasound transducer comprises a large number of transducer array elements, and then calculation of the transmit delays comprises the following steps:
obtaining the coordinates of an imaging focal point and the coordinates of a central point of any array element in the transducer array elements arranged in a two-dimensional area array by the transducer, obtaining the distance from any array element to the imaging focal point by calculation according to the coordinates of the central point and the coordinates of the imaging focal point, and obtaining the transmit delay of any array element by calculation based on the distance; and
repeatedly changing the space direction of the ultrasound beam, changing the coordinates of the imaging focal point according to the space direction, and repeating the above steps to obtain the transmit delays of all array elements.

7. The method according to claim 6, wherein the transducer array elements are divided into M sub-groups; the step of performing preprocessing and channel reduction operation based on the ASIC pre-beamforming algorithm on the reflected echo signal by the receiving circuit specifically comprises:
calculating the total delay of the reflected echo signal, and respectively performing interpolation on the reflected echo signal based on the total delay to obtain M signals by the receiving circuit of the ASIC chip;
the step of calculating the total delay of the reflected echo signal, and respectively performing interpolation on the reflected echo signal based on the total delay to obtain M signals specifically comprises: calculating a first distance from the imaging focal point to each array element in each group of array elements and a second distance from the imaging focal point to each group of central array elements, wherein the distance difference between the first distance and the second distance divided by the sound speed is a time difference, applying a delay corresponding to the time difference on the signal of each array element based on the time difference, and then adding the signals to obtain M signals;
wherein M is a natural number of greater than zero, M being equal to the number of leads of the transducer and smaller than the number of channels of the transducer.

8. The method according to claim 7, wherein the total delay comprises transmit delays and receive delays.

9. The method according to claim 8, wherein the step of performing beamforming operation and first calculation operation based on the M signals after preprocessing and channel reduction operation by the ultrasound system to obtain the gray valve and coordinate value of each point on the ultrasound beam in a certain direction specifically comprises:
delaying and adding the M signals by the ultrasound system to obtain addition data, calculating the signal delay corresponding to each channel of the transducer according to the transmission direction of the ultrasound beam, the transmit delays and the receive delays, and then delaying and adding the signals based on the signal delays; and
performing interpolation operation based on the delayed and added signals to obtain the gray value and coordinate value of each point on the ultrasound beam in a certain direction.

10. The method according to claim 9, wherein the interpolation operation and the conversion operation of the gray valves and coordinate values of all points in the Cartesian coordinate system are performed in multiple threads in a GPU (Graphic Processing Unit) or CPU (Central Processing Unit).
